(19) United States
(12) Patent Application Publication
(10) Pub. No.: US 2026/0325170 A1
(43) Pub. Date: Oct. 22, 2026

(54) VERTEBRAL BODY ACCESS DEVICE WITH AN EXIT PORT FOR IMPROVED DIRECTIONAL CONTROL

(71) Applicant: Relievant Medsystems, Inc., Edina, MN (US)

(72) Inventor: Benjamin Parker, Sunnyvale, CA (US)

(21) Appl. No.: 19/655,462

(22) Filed: Apr. 22, 2026

Related U.S. Application Data
(60) Provisional application No. 63/792,703, filed on Apr. 22, 2025.

(51) Int. Cl. A61B 17/34; A61B 17/56; A61B 17/00

(52) U.S. Cl. CPC A61B 17/3423; A61B 17/56; A61B 2017/00367; A61B 2017/564

(57) ABSTRACT

A medical device that includes an elongate shaft with a central channel and an angled port. The port comprises an angled surface oriented at a non-perpendicular angle relative to the central axis of the shaft. This angled surface engages a channeling member during rotation and movement to provide steering control at the distal end while mechanically limiting applied torque. When excessive movement or rotational force is applied, the angled geometry causes controlled disengagement of the channeling member from the port rather than device failure. The devices enable precise navigation within vertebral bodies while incorporating geometric safety features that prevent breakage of components during procedures.

### CROSS REFERENCE TO RELATED APPLICATIONS

The present application claims priority to U.S. Provisional Patent Application No. 63/792,703 filed April 22, 2025, the entire disclosure of which is incorporated by reference herein.

### TECHNICAL FIELD

Described herein are various implementations of systems and methods for accessing and treating tissue within vertebral bodies. More specifically, the disclosure relates to medical devices with angled ports that provide controlled steering and torque limitation for accessing and treating locations within vertebral bodies, for example, to treat chronic lower back pain.

### BACKGROUND

Back pain is a very common health problem worldwide and is a major cause for work-related disability benefits and compensation. At any given time, low back pain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs, including vertebral endplate degeneration or defects (e.g., pre-Modic changes). The back pain may be acute or chronic. Existing treatments for chronic back pain vary widely and include physical therapy and exercise, chiropractic treatments, injections, rest, pharmacological therapy such as opioids, pain relievers or anti-inflammatory medications, and surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

Minimally invasive approaches for treating back pain often require precise navigation within vertebral bodies. Current medical devices used to access vertebral bodies may employ channeling members that extend through ports or openings to reach treatment sites. However, these devices can face challenges with steering control and potential mechanical failure when excessive torque is applied. Traditional devices often rely on steering control which can result in material wind-up and reduced precision. Additionally, conventional ports may concentrate stress at certain points during steering, potentially leading to device failure or breakage. This creates risks during procedures, particularly if device components were to break within the vertebral body.

### SUMMARY

In Example 1, a medical device is provided comprising an elongate shaft defining a central channel extending from a proximal end to a distal end of the shaft, the shaft defining a distal port in communication with the central channel. The medical device further comprising a channeling member disposed within the central channel of the shaft and configured to extend through the port. The port comprises an angled surface oriented at a non-perpendicular angle relative to a central axis of the elongate shaft and an end surface oriented at a perpendicular angle relative to the central axis, wherein the non-perpendicular angle of the surface is configured to facilitate disengagement of the channeling member from the angled surface and facilitate engagement of the channeling member with the end surface when a force exceeds a predetermined threshold.

In Example 2, the medical device of Example 1, wherein the channeling member defines a proximal end and a curvable distal end. The distal end of the channeling member is movable between a first position, at which the distal end of the channeling member is disposed within the central channel proximal of the port, and a second position, at which the distal end of the channeling member is disposed exterior of the central channel and the port. The channeling member is configured to channel through cancellous bone in a vertebral body.

In Example 3, the medical device of any one of Examples 1 or 2, wherein the channeling member comprises a curvable distal portion configured to deflect within a vertebral body when extended through the port.

In Example 4, the medical device of any one of Examples 1-3, wherein the angled surface is configured to distribute rotational and axial forces to change the point of contact between the channeling member and the elongate shaft.

In Example 5, the medical device of any one of Examples 1-4, wherein the angled surface is further configured to provide controlled directional movement of the channeling member.

In Example 6, the medical device of any one of Examples 1-5, wherein the non-perpendicular angle of the surface is configured to limit or increase torque transmission to the channeling member based on the geometric relationship between the angled surface and the channeling member.

In Example 7, the medical device of any one of Examples 1-6, wherein the non-perpendicular angle is configured to convert a portion of force into an axial force and/or rotational force component that facilitates controlled disengagement of the channeling member.

In Example 8, the medical device of any one of Examples 1-7, wherein the port is configured to enable complete withdrawal of the medical device from a vertebral body upon disengagement of the channeling member.

In Example 9, the medical device of any one of Examples 1-8, wherein the non-perpendicular angle is between 10 degrees and -10 degrees relative to the central axis of the elongate shaft.

In Example 10, the medical device of any one of Examples 1-9, wherein the angled surface is configured to maintain engagement with the channeling member when applied force is below the predetermined threshold; gradually cause a contact point between the channeling member and the angled surface to shift in a distal direction as applied force approaches the predetermined threshold; and enable re-engagement of the channeling member when applied force is reduced below the predetermined threshold.

In Example 11, the medical device of any one of Examples 1-10, wherein the angled surface extends circumferentially around a portion of the elongate shaft to define a controlled engagement region.

In Example 12, the medical device of any one of Examples 1-11, wherein the angled surface transitions steering control from the proximal end to the distal end of the elongate shaft.

In Example 13, the medical device of Example 12, wherein the distal steering control reduces torsional stress accumulation of the channeling member within the central channel.

In Example 14, the medical device of any one of Examples 1-13, wherein the port comprises a geometry that provides increasing mechanical resistance as the force approaches the predetermined threshold.

In Example 15, the medical device of any one of Examples 1-14, wherein the angled surface comprises a geometric configuration that maintains directional control while preventing structural failure of the channeling member.

In Example 16, a medical device is provided comprising an elongate shaft defining a central channel extending from a proximal end to a distal end of the shaft, the shaft defining a distal port in communication with the central channel. The medical device further comprising a handle disposed at the shaft proximal end, a channeling member disposed within the central channel of the shaft and configured to extend through the port. The port comprises an angled surface oriented at a non-perpendicular angle relative to a central axis of the elongate shaft and an end surface oriented at a perpendicular angle relative to the central axis, wherein the non-perpendicular angle of the surface is configured to facilitate disengagement of the channeling member from the angled surface and facilitate engagement of the channeling member with the end surface when a force exceeds a predetermined threshold.

In Example 17, the medical device of Example 16, wherein the channeling member defines a proximal end and a curvable distal end. The distal end of the channeling member is movable between a first position, at which the distal end of the channeling member is disposed within the central channel proximal of the port, and a second position, at which the distal end of the channeling member is disposed exterior of the central channel and the port. The channeling member is configured to channel through cancellous bone in a vertebral body.

In Example 18, the medical device of Example 16, wherein the channeling member comprises a curvable distal portion configured to deflect within a vertebral body when extended through the port.

In Example 19, the medical device of Example 16, wherein the angled surface is configured to distribute rotational and axial forces to change the point of contact between the channeling member and the elongate shaft.

In Example 20, the medical device of Example 16, wherein the angled surface is further configured to provide controlled directional movement of the channeling member.

In Example 21, the medical device of Example 16, wherein the non-perpendicular angle of the surface is configured to limit or increase torque transmission to the channeling member based on the geometric relationship between the angled surface and the channeling member.

In Example 22, the medical device of Example 16, wherein the non-perpendicular angle is configured to convert a portion of force into an axial force and/or rotational force component that facilitates controlled disengagement of the channeling member.

In Example 23, the medical device of Example 16, wherein the port is configured to enable complete withdrawal of the medical device from a vertebral body upon disengagement of the channeling member.

In Example 24, the medical device of Example 16, wherein the non-perpendicular angle is between 10 degrees and -10 degrees relative to the central axis of the elongate shaft.

In Example 25, the medical device of Example 16, wherein the angled surface is configured to maintain engagement with the channeling member when applied force is below the predetermined threshold; gradually cause a contact point between the channeling member and the angled surface to shift in a distal direction as applied force approaches the predetermined threshold; and enable re-engagement of the channeling member when applied force is reduced below the predetermined threshold.

In Example 26, the medical device of Example 16, wherein the angled surface extends circumferentially around a portion of the elongate shaft to define a controlled engagement region.

In Example 27, the medical device of Example 16, wherein the angled surface transitions steering control from the proximal end to the distal end of the elongate shaft.

In Example 28, the medical device of Example 27, wherein the distal steering control reduces torsional stress accumulation of the channeling member within the central channel.

In Example 29, the medical device of Example 16, wherein the port comprises a geometry that provides increasing mechanical resistance as the force approaches the predetermined threshold.

In Example 30, the medical device of Example 16, wherein the angled surface comprises a geometric configuration that maintains directional control while preventing structural failure of the channeling member.

In Example 31, a system for treating tissue within a vertebral body is provided. The system comprises an elongate shaft defining a central channel extending from a proximal end to a distal end of the shaft, the shaft defining a port in communication with the central channel, the shaft further comprising a distal tip. The system further comprises a handle disposed at the shaft proximal end, and a channeling member disposed within the central channel of the shaft and configured to extend through the port wherein the channeling member defines a proximal end and a curvable distal end. The distal end of the channeling member is movable between a first position, at which the distal end of the channeling member is disposed within the central channel proximal of the port, and a second position, at which the distal end of the channeling member is disposed exterior of the central channel and the port, and the channeling member is configured to channel through cancellous bone in the vertebral body. The port comprises an angled surface oriented at a non-perpendicular angle relative to a central axis of the elongate shaft, wherein the angled surface is configured to engage the channeling member during rotational movement of the shaft to translate rotational force to the channeling member. The non-perpendicular angle of the surface is configured to facilitate disengagement of the channeling member from the port when the rotational force exceeds a predetermined threshold.

In Example 32, the system of Example 31, wherein the angled surface proximate to the distal end of the shaft is configured to reduce mechanical stress concentration on the channeling member during rotational movement.

In Example 33, the system of Example 31, wherein the non-perpendicular angle is configured to convert a portion of rotational force into an axial force component that facilitates controlled disengagement of the channeling member.

In Example 34, the system of Example 31, wherein the angled surface transitions steering control from the proximal end to the distal end of the elongate shaft to reduce torsional stress accumulation of the channeling member within the central channel.

In Example 35, a method of treating tissue within a vertebral body of a patient is provided. The method comprises providing a medical device comprising a cannula defining a central channel extending from a proximal end to a distal end of the cannula, the cannula defining a port proximate the distal end and in communication with the central channel, the port comprising an angled surface oriented at a non-perpendicular angle relative to a central axis of the cannula and an end surface oriented at a perpendicular angle relative to the central axis. The medical device further comprises a handle disposed at the cannula proximal end, the handle comprising a first actuator, a stylet disposed within the central channel of the cannula, the stylet defining a proximal end and a distal end, wherein the stylet is coupled with the first actuator and axially movable, in response to actuation of the first actuator, such that the distal end of the stylet is advanceable out of the port and retractable into the central channel of the cannula. The method comprises inserting the cannula of the medical device through cortical bone of the vertebral body into a cancellous bone region of the vertebral body; actuating the first actuator to advance the distal end of the stylet out of the port; rotating the cannula to steer the stylet within the vertebral body, wherein the angled surface of the port engages the stylet to translate rotational force to the stylet, and the non-perpendicular angle facilitates disengagement of the stylet from the angled surface when rotational force exceeds a predetermined threshold; creating a channel within the vertebral body using the stylet; advancing a treatment device through the channel to a treatment location within the vertebral body; and delivering treatment to the tissue within the vertebral body via the treatment device.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

Having thus described embodiments of the present disclosure in general terms, reference will now be made to the accompanying drawings, which are not necessarily drawn to scale, and wherein:

FIG. 1 is a schematic illustration of vertebral levels and vertebrae that can be treated by systems and methods consistent with various aspects of the present disclosure.

FIG. 2 is a side view of a medical device, consistent with various aspects of the present disclosure.

FIG. 3A is a close-up, side view of the introducer, consistent with various aspects of the present disclosure.

FIG. 3B is a top view of a port of the introducer of FIG. 3A with angled surfaces for torque control, consistent with various aspects of the present disclosure.

FIG. 3C is a side view of an alternative port of the introducer of FIG. 3B, consistent with various aspects of the present disclosure.

FIG. 3D is a top view of an alternative port of the introducer of FIG. 3A, consistent with various aspects of the present disclosure.

FIG. 3E is a side view of the introducer showing a medical component extending through the angled port of FIG. 3B at a first circumferential position, consistent with various aspects of the present disclosure.

FIG. 3F is a front view of the port showing the medical component extending through the angled port of FIG. 3B at the first circumferential position, consistent with various aspects of the present disclosure.

FIG. 3G is a side view of the introducer showing the medical component extending through the angled port of FIG. 3B at a second circumferential position, consistent with various aspects of the present disclosure.

FIG. 3H is a front view of the introducer showing the medical component extending through the angled port of FIG. 3B at the second circumferential position, consistent with various aspects of the present disclosure.

FIG. 3I is a side view of an alternative port configuration with a converging angled surface showing a medical component extending through the alternative port configuration, consistent with various aspects of the present disclosure.

FIG. 3J is a side view of an alternative port configuration with parallel surfaces showing a medical component extending through the alternative port configuration, consistent with various aspects of the present disclosure.

FIG. 4 is a flowchart of a method of accessing and treating tissue within a vertebral body, consistent with various aspects of the present disclosure.

FIGS. 5A-5E are a series of top views illustrating operations of accessing and treating tissue within a vertebral body, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Embodiments described herein provide improved medical devices and methods that address many of the above challenges associated with vertebral access and treatment. Various embodiments include medical devices with angled ports that enable improved distal steering control while incorporating mechanical safeguards against excessive torque application.

The devices and methods disclosed herein include medical devices with ports having angled surfaces that engage channeling members and other medical instruments. The angled geometry provides transfers in steering control from the proximal end to the distal end of the device and reduces material wind-up and improving precision. The angled geometry of the port also creates a mechanical torque-limiting function that causes controlled disengagement of the channeling member or other medical instruments when excessive rotational force and/or movement is applied, preventing breakage within the vertebral body.

Example embodiments may include various port angles and geometries to optimize the balance between steering control and safety features. The angled port configuration enables simpler and more reliable vertebral access compared to complex mechanical solutions like slip clutches or torque limiters, while maintaining effectiveness and safety.

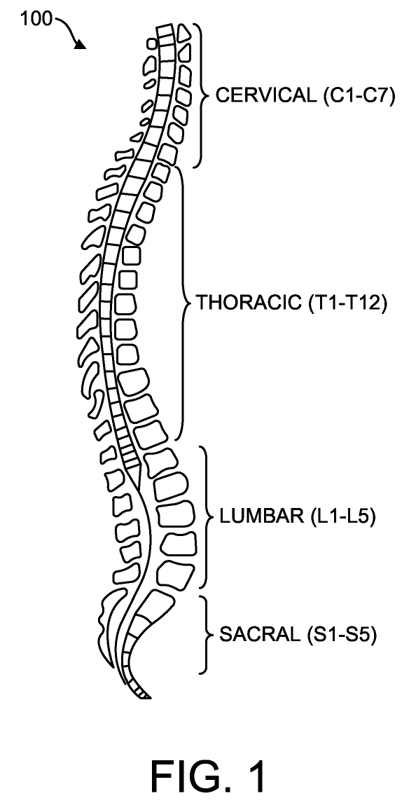

FIG. 1 is a schematic illustration of a human spine 100 including vertebral levels and vertebrae that can be treated by systems and methods consistent with various aspects of the present disclosure. Treatment procedures may include modulation of nerves within or surrounding bones. The terms “modulation” or “neuromodulation”, as used herein, shall be given their ordinary meaning and shall also include ablation, permanent denervation, temporary denervation, disruption, blocking, inhibition, electroporation, therapeutic stimulation, diagnostic stimulation, inhibition, necrosis, desensitization, or other effect on tissue. Neuromodulation shall refer to modulation of a nerve (structurally and/or functionally) and/or neurotransmission. Modulation is not necessarily limited to nerves and may include effects on other tissue, such as tumors or other soft tissue.

Embodiments described herein are directed to systems and methods for modulating nerves within or adjacent (e.g., surrounding) bone. In some embodiments, an intraosseous nerve (e.g., basivertebral nerve (BVN)) within a vertebral body of the spine is modulated for treatment or prevention of chronic back pain. The vertebral body may be located in any level of the vertebral column (e.g., cervical, thoracic, lumbar, and/or sacral). Multiple vertebral bodies may be treated in a single visit or procedure (simultaneously or sequentially). The multiple vertebral bodies may be located in a single spine segment or in different spine segments. Examples of the former are two adjacent vertebral bodies in the sacral spine segment (e.g., S1 and S2) or lumbar spine segment (e.g., L3, L4 and/or L5) or thoracic spine segment or cervical spine segment), and an example of the latter is an L5 vertebra in the lumbar spine segment and an S1 vertebra in the sacral spine segment. Intraosseous nerves within bones other than vertebral bodies may also be modulated. For example, nerves within a humerus, radius, femur, tibia, calcaneus, tarsal bones, hips, knees, and/or phalanges can be modulated.

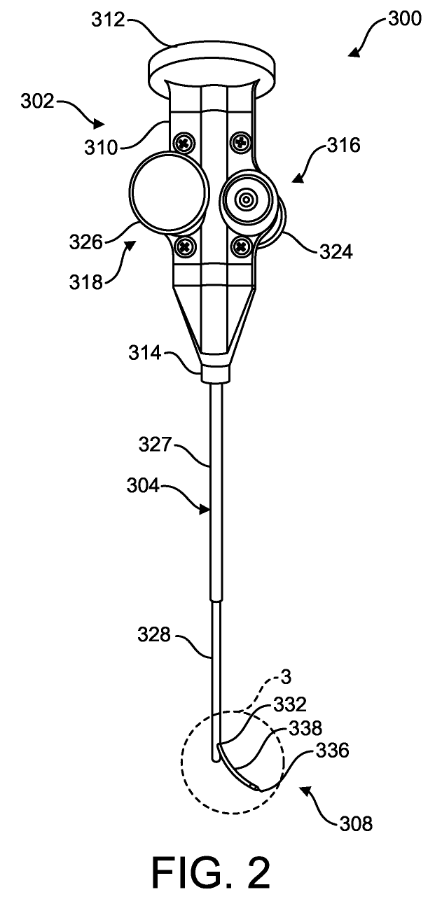

FIG. 2 shows a medical device 300 for accessing and creating channels within a vertebral body. As shown in FIG. 2, the medical device 300 includes a handle 302, an introducer shaft 304, and extendable components 308. The medical device 300 is configured to access the vertebral body, position therapy components adjacent the treatment tissue (e.g., the basivertebral nerve) and deliver sufficient treatment such as radiofrequency energy to the nerve to modulate (e.g., ablate) the nerve so as to reduce or eliminate a patient’s pain.

As shown, the handle 302 includes a body 310, including a proximal end 312 and a distal coupling feature 314, and it further includes a first actuator 316 and a second actuator 318. In the exemplary embodiment of FIG. 2, the proximal end 312 is substantially disc-shaped and has sufficient strength to serve as a “malletable” surface. In other words, during use, the physician may strike the proximal end 312 with a rubber mallet with sufficient force to drive the distal end of the introducer shaft 304 through the cortical bone of the vertebral body. The handle further includes a coupling feature 314 to engage with the introducer shaft 304. The actuators 316 and 318 and coupled to the extendable components 308 and operate to drive these components out of the introducer shaft 304 and toward the basivertebral nerve. Each of the actuators 316 and 318 include corresponding knobs 324 and 326, which are configured to be readily manipulated (e.g., rotated) by the physician.

As shown in FIG. 2, the introducer shaft 304 extends distally from the coupling feature 314 of the handle 302 in a longitudinal direction. The introducer shaft 304 has a thicker proximal section 327 joined to a thinner distal section 328. A proximal portion of the proximal section 327 is disposed inside and coupled to the handle 302. The introducer shaft 304 is an open-ended hollow shaft that includes a bore or lumen, which extends from the proximal end to a lateral or radial opening (or port) 332, which is sized and shaped to allow the extendable components 308 to exit therethrough. In some embodiments, the proximal section 327 has a diameter between about 4.7 millimeters (mm) to about 6.3 mm, while the distal section 328 has a diameter between about 2.5 mm to about 4.4 mm. In some embodiments, the diameter of the distal section 328 is about 3.048 mm.

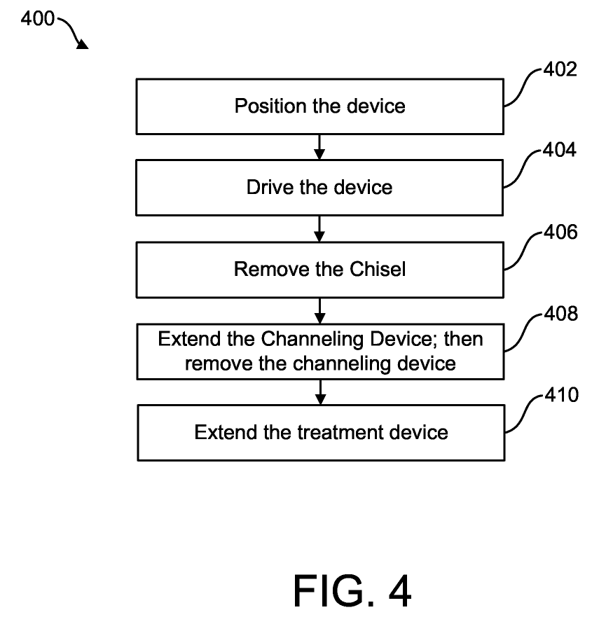

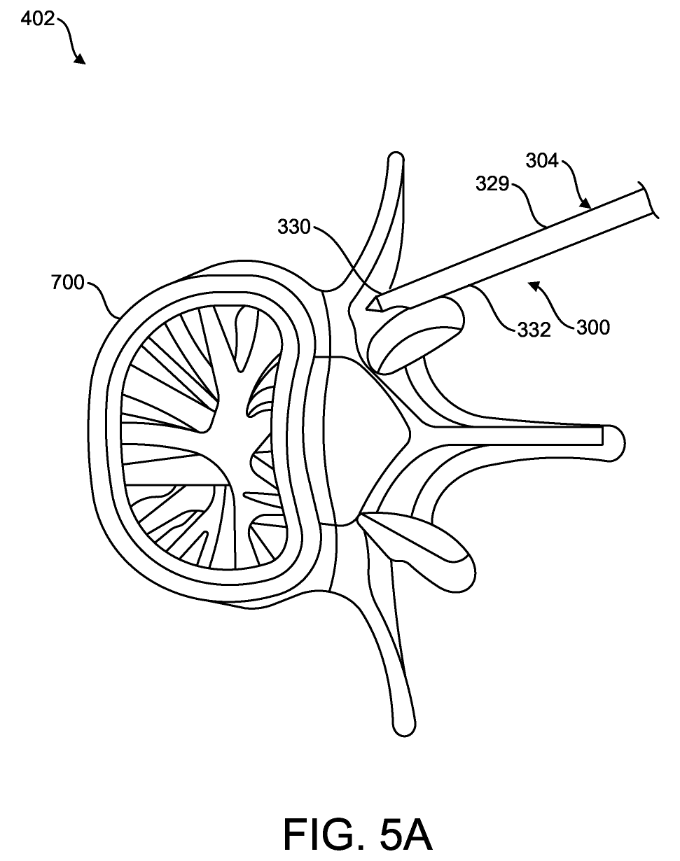

The extendable components 308 include proximal portions disposed inside the handle 302 that are coupled to the actuators 316, 318, respectively. These proximal portions extend distally through the bore in the shaft to a location near lateral opening 332. The extendable components 308 may include various elements such as stylets, channeling devices, probes, or other components configured to extend through the lateral opening 332 for creating channels or accessing treatment locations within the vertebral body. As shown in FIG. 2, the extendable components 308 are in their extended positions, i.e., the physician has manipulated the actuators 316, 318 an amount sufficient to advance the extendable components 308 out of the introducer shaft 304 through the lateral opening 332. When the physician has manipulated the actuators 316, 318 to retract the extendable components 308, the extendable components 308 may be fully retracted (through the lateral opening 332) such that they are fully nested within the bore of the shaft 304 (as shown in FIGS. 4A and 4B).

The lateral opening 332 may also comprise different configurations wherein one configuration may include geometric features including angled surfaces that engage with the extendable components 308 during use. These surfaces may be oriented at non-perpendicular angles relative to the central axis 375 of the shaft, enabling both enhanced steering control and mechanical safety features. During use, the physician can manipulate the components through the actuators 316, 318 to cause them to extend from the introducer shaft 304 toward the treatment location. The angled surfaces of the lateral opening may provide direct steering control at the distal end of the device while incorporating geometric features that limit applied torque to prevent component failure during the procedure.

The extendable components 308 may include a channeling member 338 and a channeling distal tip 336, each coupled at their proximal ends to actuators 316, 318, respectively. The channeling member 338 and channeling distal tip 336 work together to create channels within the vertebral body, with the channeling distal tip 336 configured to define an initial path and the channeling member 338 configured to follow and potentially enlarge this path. The geometric configuration of the lateral opening 332 may enable precise control of both components during advancement and steering, while providing mechanical safeguards through its angled surfaces.

In some embodiments, the extendable components 308 take different forms depending on the specific procedural requirements. For example, the channeling member 338 and channeling distal tip 336 are configured as a curved stylet assembly for creating curved channels within the vertebral body. In some embodiments, the components include a J-stylet for defining curved paths, straight stylets for linear channels, treatment probes for delivering energy or other therapies, or various combinations thereof. The lateral opening and the angled surfaces are configured to provide controlled steering and torque limitation regardless of the specific component configuration being used. This enables the device to accommodate different procedural approaches while maintaining the safety and control benefits provided by the angled port geometry.

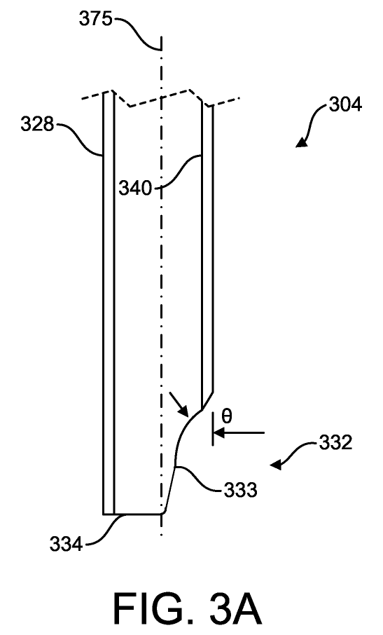

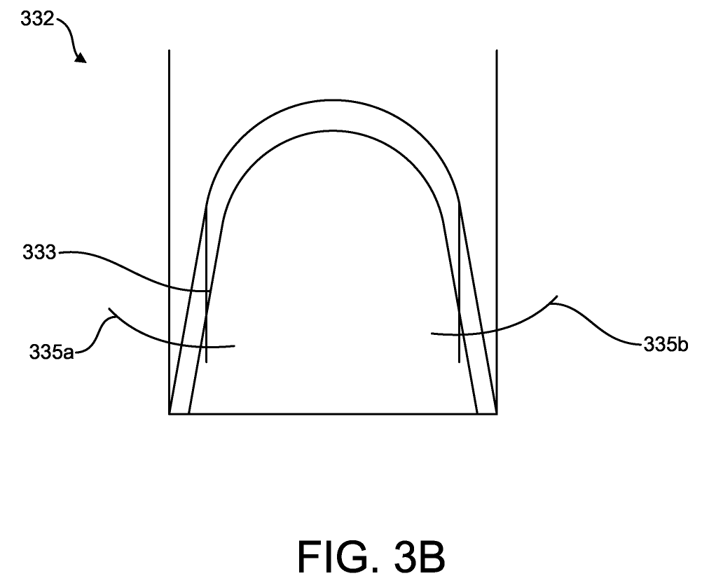

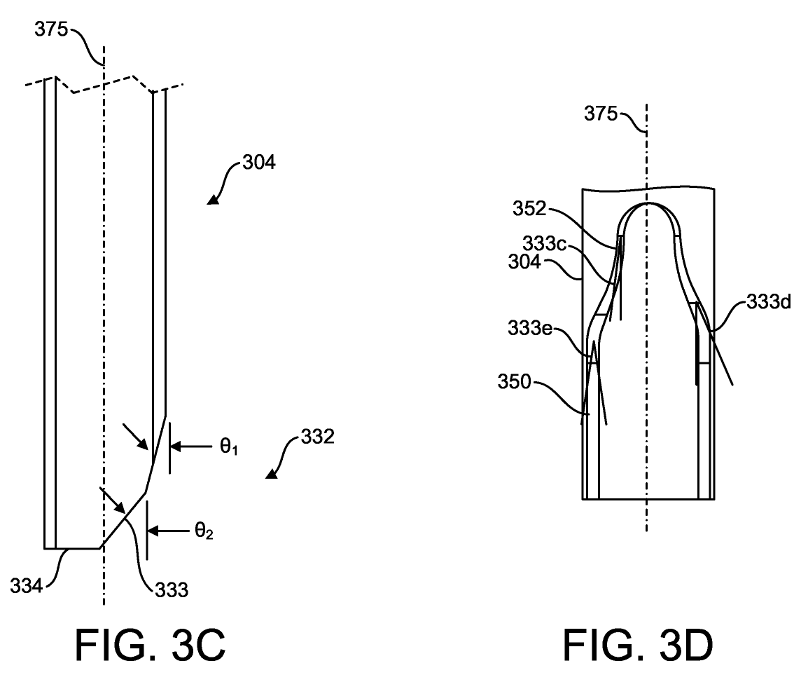

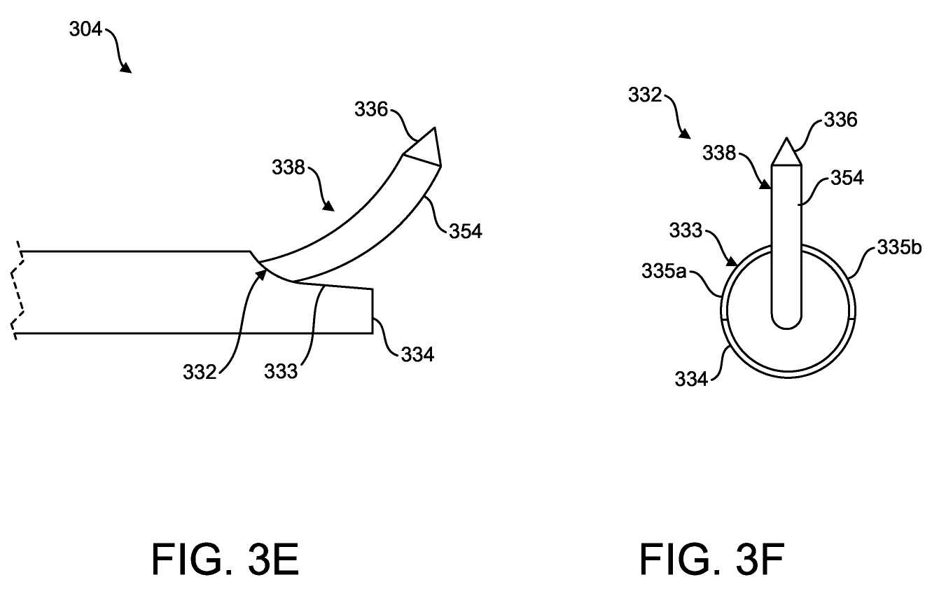

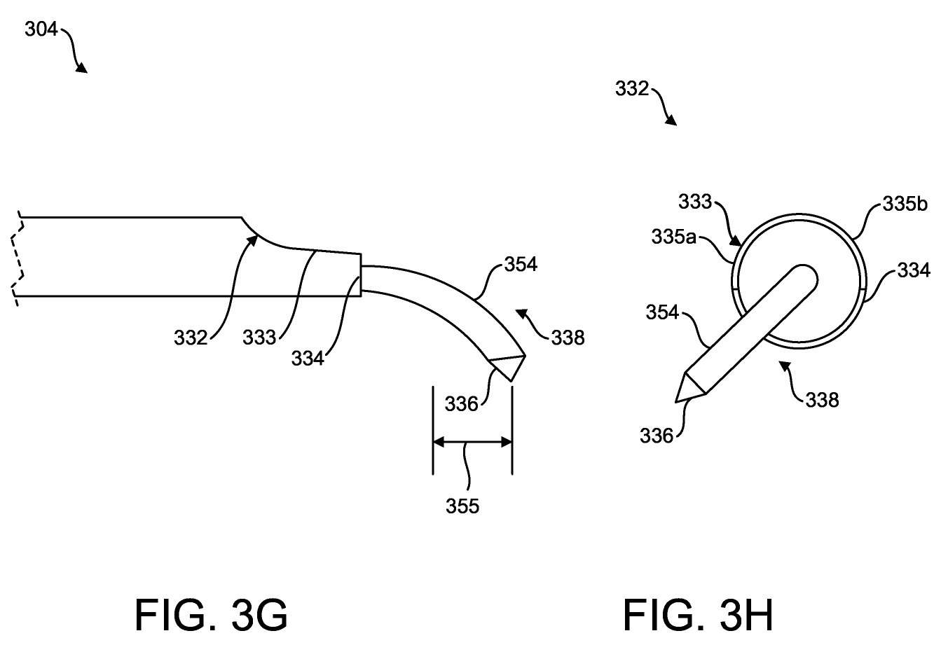

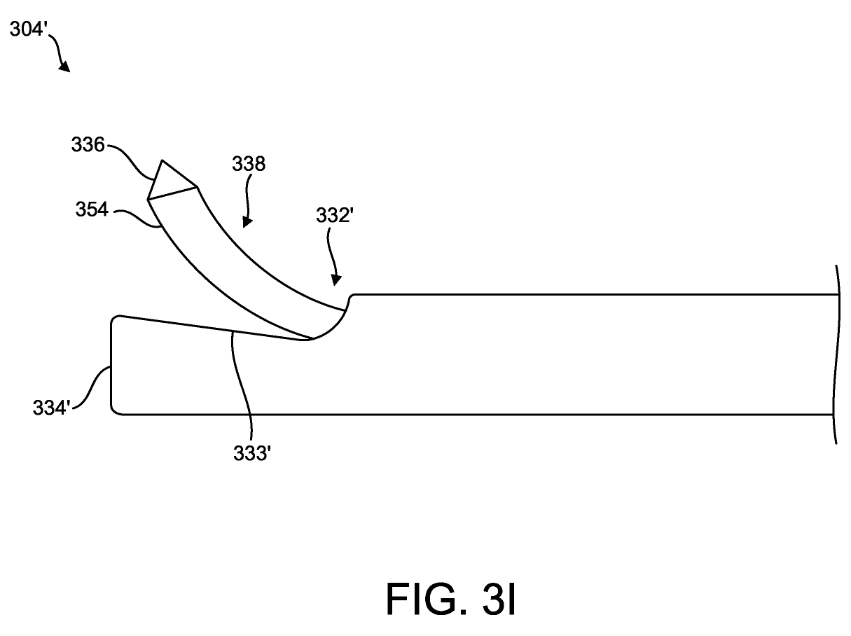

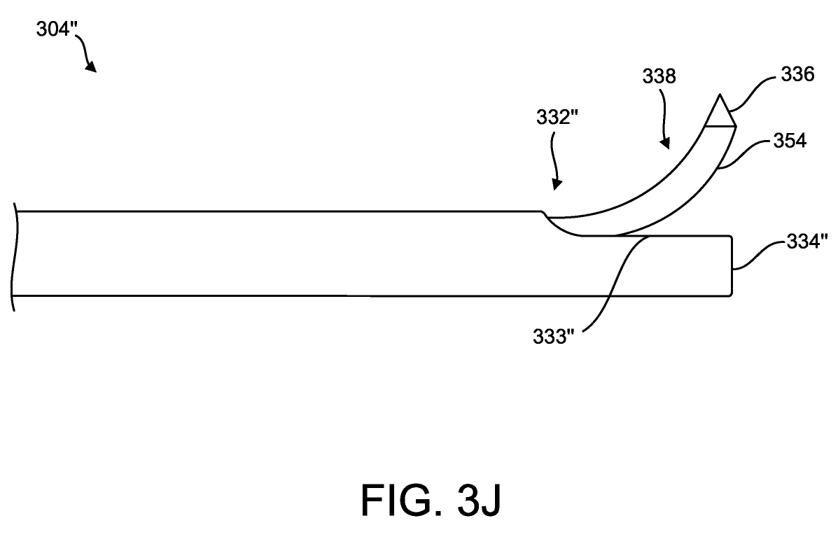

FIGS. 3A-3H show detailed views of various aspects of the distal portion of the introducer shaft 304 indicated by the phantom circle 3 in FIG. 2. These figures illustrate various aspects of the introducer 304, particularly focusing on the port configurations that enable controlled steering and torque limitation.

FIG. 3A is a cross-sectional view of the introducer shaft 304. The introducer shaft 304 includes an inner surface 340 defining the longitudinally extending bore and is breached at the distal end by the lateral opening 332 (or port). The opening 332 creates a pathway for the extendable components to exit from the bore to the exterior of the introducer shaft 304. The cross-sectional view illustrates how the inner surface 340 forms a continuous channel that accommodates the movement of the extendable components while the lateral opening 332 provides a positioned exit port for directional deployment. The inner diameter of the bore and the dimensions of the lateral opening 332 can also be selected such to enable smooth passage and controlled deployment of the extendable components while maintaining sufficient structural rigidity of the introducer shaft 304 for advancement through tissue. The cross-sectional view also illustrates angled surface 333, which forms a boundary of the lateral opening 332. This angled surface 333 is oriented at a non-perpendicular angle relative to the central axis 375 of the introducer shaft 304, creating a geometric interface that influences the deployment and steering of extendable components 308. Adjacent to the angled surface 333 is the disengagement surface 334, which completes the distal boundary of the lateral opening 332. Together, these surfaces create a port configuration that provides both directional control and torque limitation functionality.

FIG. 3B illustrates the port 332 configuration within the introducer shaft 304, wherein the port 332 comprises an angled surface 333 disposed at a non-perpendicular angle relative to the central axis 375 of the shaft 304 and a disengagement surface 334 at a perpendicular angle relative to the central axis 375 of the shaft 304. This surface defines a geometric configuration that provides control over torque transmission and component steering through mechanical principles rather than additional mechanical elements. In the illustrated embodiment, the angled surface results in lateral angles 335a and 335b that are the same amount and extend laterally outward in opposite directions, for example, between about 5 and about 15 degrees or at about 10 degrees from the central axis 375 of the introducer 304.

The angled surface 333 extends as a cylindrical section across a portion of the elongate shaft that includes part of the distal end to define a controlled engagement region. The disengagement surface 334 completes the distal end of the introducer 304 to define a disengagement region. This controlled engagement region provides a geometric interface where the channeling member 308 interacts with the port surfaces during rotation and movement, enabling both steering control and progressive disengagement under excessive rotational force conditions. The disengagement region provides a geometric interface where the channeling member 308 cannot be rotated by further rotation of the introducer 304 to limit the amount of rotational force that can be imparted to the channeling member 308.

The angled surface 333 creates a force transformation system through their geometric relationship with extendable components 308 (shown in FIG. 2) (e.g. J-stylet, probes, etc.) that extend through port 332. When rotational force or lateral movement is applied to the introducer 304 or to the extendable components 308, these angles 335a and 335b resolve the force into both rotational and axial components. The ratio between these force components varies based on the specific angles 335a and 335b selected. If excessive force is applied in any direction, such as when encountering dense bone or attempting aggressive directional changes, the angled surface 333 begins converting these increased forces into relative axial movement between the extendable components 308 and the introducer 304 (i.e., the extendable component 308 will be forced distally outwards from the introducer 304 and/or the introducer 304 will be forced proximally outwards from the patient’s body). The extendable component 308 starts to disengage from the angled surface 333 rather than experiencing sufficient mechanical stress that could lead to failure.

The mechanical behavior of the port 332 may vary across different angular configurations. With smaller angles, the angled surface 333 maintains greater contact with the extendable component 308 during rotation and movement, providing enhanced steering control while still maintaining disengagement capabilities at higher torque values. As the angles increase there may be a result in earlier disengagement under rotation and/or movement and requiring less total force to initiate the disengagement. During operation, as rotational force and movement is applied to the introducer 304, the angled surface 333 engage with the extendable component in a manner that translates the movement into controlled steering forces at the distal end of the device. This geometric arrangement transfers steering control from the proximal handle to the port 332 location.

The port 332 configuration with angled surface 333 maintains its force conversion and safety features across a range of medical instruments that may be advanced through the port. Whether deploying cutting instruments, treatment delivery devices, visualization tools, or specialized probes, the geometric principles governing force distribution and controlled disengagement remain consistent. The angled surface 333 accommodates these various instruments while providing the same mechanical advantages - steering control at the distal end and prevention of component failure through controlled disengagement when excessive force is applied. This versatility enables the port configuration to serve multiple procedural requirements while maintaining consistent safety and control characteristics across different tool types. In some embodiments, the angled surface 333 employ compound angles or curved surfaces that provide varying degrees of force conversion based on the position of the extending component within the port (as shown in FIGS. 3C and 3D).

The angled surface 333 may be configured within a range of angles relative to the central axis 375, from relatively shallow angles to steeper angles. In some embodiments the angles may range from approximately 10 degrees to approximately -10 degrees. In some embodiments, the port opening 332 has an axial length ranging from about 0.2 to about 0.4 mm. The selection of specific angles 335a and 335b determines the force transmission characteristics and steering behavior of the device.

For smaller diameter devices, shallower angles 335a and 335b between 5-30 degrees may provide optimal steering control by maintaining primary engagement during fine movements. These angles enable for precise adjustments while still incorporating disengagement features when excessive force is applied in any direction. As device diameter increases, steeper angles may be employed to accommodate smaller rotational forces while maintaining controlled movement characteristics.

The port 332 geometry's effect on component 308 behavior varies with both angle selection and movement type. During advancement, the angled surface 333 guide the extendable component 308 along a controlled path while enabling for directional adjustments. During rotation or movement, these same surfaces convert excessive forces into outward movement. When retracting the extendable component 308, the angled surface 333 maintain engagement until deliberate disengagement is desired.

The force decomposition created by angles 335a and 335b occurs through trigonometric relationships. The total input force divides into tangential and axial components based on the sine and cosine of the surface angles. This mathematical relationship provides predictable and repeatable behavior across different operating conditions.

When rotational force is applied to the introducer 304 or extendable component 308, the angled surface 333 converts a portion of this rotational force into axial force through their geometric configuration. At rotational forces below a predetermined threshold, the majority of force remains rotational, enabling precise steering control. As rotational force increases beyond this threshold, a greater portion converts to axial force, facilitating controlled disengagement of the extendable component 308 from the port 332. The predetermined threshold may depend on multiple factors including the specific angles 335a and 335b, material properties of the components, and surface characteristics of the port 332. For a given configuration, this threshold represents the point at which sufficient axial force develops to initiate component disengagement. Below this threshold, the extendable component 308 maintains consistent engagement with the angled surface 333 for reliable steering performance.

The threshold optimization may also involve balancing these geometric relationships against clinical requirements. The threshold may be optimized by selecting angles 335a and 335b that produce desired force at specific torque inputs. For example, configuring the angles to maintain 80% rotational force at normal operating torques while increasing axial force conversion at higher torques may provide both steering control and safety disengagement.

Furthermore, the force conversion that creates the disengagement occurs progressively rather than suddenly, creating a predictable transition as forces approach and exceed the threshold. This progressive conversion prevents abrupt disengagement while providing tactile feedback to the operator through increasing resistance. The rate of conversion varies with the angles 335a and 335b, with larger angles producing more rapid conversion of rotational force to axial force.

When a physician manipulates the extendable component 308 through port 332 during navigation within the vertebral body, the angled surface 333 initially maintain primary engagement to enable controlled movement. The physician may apply various forces - rotational force for steering, axial force for advancement or retraction, or combinations of forces during complex maneuvers. If excessive force is applied rotationally and/or axially in any direction, such as when encountering dense bone or attempting aggressive directional changes, the angled surface 333 begins converting these increased forces into outward movement. At a certain threshold - determined by the specific angle geometry - the extendable component 308 starts to disengage from the angled surface 333 rather than experiencing mechanical stress that could lead to failure. This progressive disengagement happens as a gradual reduction in resistance that provides tactile feedback indicating that the intended force range in any direction has been exceeded. When the applied force is reduced, the extendable component 308 is enabled to re-engage with the angled surface 333 and continue controlled navigation. This geometric interaction prevents component failure while maintaining procedural continuity, regardless of the type or direction of force applied. The angled surface 333 distribute contact forces and reduces local stress concentrations that could lead to material failure.

After disengagement occurs, the port 332 geometry enables recovery and return to normal operation. For instance, when the physician reduces applied forces, the physician would realign the extendable component 308 with the angled surface 333 to reestablish proper engagement. This would involve repositioning the extendable component 308 relative to the port 332 by retracting the component 308 and readvancing the component 308. Once properly realigned with the angled surface 333, the extendable component 308 may be manipulated again with controlled steering through the geometric features of the port 332.

The recovery characteristics remain consistent across different reasons for of disengagement. Whether the extendable component 308 disengages due to excessive rotational force, axial force, or combined forces, the geometric relationship between the angled surface 333 and the extendable component 308 facilitates the same reliable return to engagement. This predictable recovery behavior enables physicians to maintain procedural flow even after encountering situations requiring disengagement, as no additional steps or tools are needed to resume normal operation.

In some embodiments where angles 335a and 335b are equal, the force distribution remains symmetric during clockwise and counterclockwise rotation of the introducer 304. Symmetric angles 335a 335b create balanced steering characteristics in both rotational directions. In some embodiments, different values may be utilized for angles 335a and 335b to achieve asymmetric force distribution for specialized steering requirements.

In some embodiments, material selection for the introducer shaft 304 and the extendable components 308 affects the interaction between the angled surfaces and extendable components. Surface hardness, friction coefficients, and wear characteristics influence both steering control and disengagement behavior. Materials may be selected to optimize these properties while maintaining biocompatibility requirements.

Surface treatments or coatings may modify the interaction between angles 335a and 335b and the extendable components 308. These treatments can alter friction coefficients to better steering control and disengagement characteristics while maintaining the underlying geometric force relationships created by the angular surfaces.

Manufacturing considerations for the port 332 may include maintaining precise angular tolerances to ensure consistent performance. The angled surface 333 can go through controlled machining and/or grinding processes to achieve specified angles within acceptable variation ranges. Surface finish requirements ensure proper interaction with extendable components.

Furthermore, the response to varying movement speeds also demonstrates rate-dependent behavior. At lower movement speeds, the force conversion between rotational and axial components follows predicted geometric relationships closely. However, as movement speed increases, dynamic effects including momentum and material characteristics may become of concern in the system's behavior.

The rate of rotation and movement may influence system behavior differently depending on material composition. With metal components, the force-displacement relationship may remain relatively consistent across different rates of applied force. The geometric principles governing disengagement apply similarly at different rotational speeds with metal components, maintaining predictable thresholds. For implementations using polymer components, rate-dependent behavior may be different, with rapid movements potentially altering the effective disengagement threshold due to material properties. The design may be accommodated for different accounts for these material-specific behaviors to maintain consistent performance across various operating conditions.

While specific angle selections may be optimized for particular component sizes, the fundamental force relationships remain applicable across different diameter ranges. The geometric principles governing the port 332 configuration maintain consistent relationships across different devices. The ratio between port dimensions and component diameters influences force transmission characteristics while maintaining the fundamental mechanical principles. Unlike mechanical torque limiters or slip clutches that require additional components and assembly, the angled surface 333 achieve torque limitation and/or torque increase through the geometry of the introducer 304 and the extendable components 308 alone. This reduces device complexity while providing comparable functional benefits through the natural behavior of the angular geometry. The controlled disengagement behavior prevents component breakage that could require leaving pieces within the anatomy. This geometric approach maintains device integrity throughout normal operation and potential emergency situations.

In some embodiments, the port 332 may include curved transitions between the angled surfaces and the central channel to modify force transmission characteristics, as shown in FIG. 3D.

In some embodiments, the port 332 may accommodate various extendable components 308 for different procedural applications. In vertebral body procedures, the extendable component 308 may comprise a straight stylet for creating linear channels, a curved stylet for generating curved paths, or specialized tips and probe for particular tissue interactions and treatments devices. Each type of device maintains the benefits of controlled steering and torque limitation and/or increase provided by the angled surface 333.

In some embodiments, the port 332 may be configured to work with a series of sequentially deployed components. For example, an initial channeling component may create a path, followed by a treatment device delivered through the same port utilizing the same angled surface geometry for controlled positioning.

In some embodiments, the introducer 304 may include additional features that complement the port 332 functionality. These may include alignment markers for port orientation, depth indicators for component advancement, or surface treatments that enhance visualization under imaging.

In some embodiments, various handle 302 configurations may be employed to optimize control of components extending through the port 332. These may include different actuator mechanisms, rotational stops, or feedback systems that complement the mechanical feedback provided by the angled surface 333.

FIG. 3C illustrates a side view of an alternative configuration of the port 332 of introducer shaft 304. The port 332 features an angled surface 333 with a geometry that changes angle along its length. The angled surface 333 begins at a first angle θ₁ relative to the central axis 375 of the shaft 304, then transitions to a second angle θ₂.

The side view demonstrates how the angled surface 333 creates a graded interface enabling components 308 to extend through the port 332. As a component 308 moves along this surface 333, it initially encounters the section defined by angle θ₁, which provides force decomposition. When the component progresses to the section defined by angle θ₂, it experiences another force decomposition. This progressive change in angles influences how forces are transmitted through the port 332 during operation.

For instance, when rotational or lateral movement is applied, the component 308 makes initial contact at the proximal portion of the angled surface 333. As force increases, the contact point migrates along the surface 333, first experiencing the force distribution defined by angle θ₁, then transitioning to the force distribution defined by angle θ₂. With continued force application, the contact point eventually reaches the disengagement surface 334, at which point rotational forces can no longer be effectively transmitted to the extendable component 308. This graduated angle arrangement enables for force management providing effective steering control at different force inputs while ensuring component protection through disengagement at higher force thresholds. central axis 375

FIG. 3D illustrates an alternative port opening 350, which is a lateral opening through the wall of the introducer shaft 304. In a side projection of the lateral opening 350, the lateral opening 350 has a bottle shape with a narrower neck region 352 at the proximal end of the lateral opening 350. This configuration also incorporates multiple angled surfaces 333c, 333d, and 333e, each oriented at different angles relative to the central axis 375 of the introducer shaft 304. Surface 333e forms a relatively shallow angle at the proximal portion of the opening 350, providing initial component guidance with minimal resistance during deployment. Surface 333d presents a steeper angle as it transitions toward the distal portion, increasing the rate of force conversion between rotational and lateral vectors.

Surface 333c completes the upper boundary of the opening 350, providing a boundary surface that influences the component's interaction with the port. The transition from shallower to steeper angles within this configuration can create progressive disengagement during component operation. When extendable component 308 is advanced through the port, it initially contacts the shallow-angled surface 333e. As rotation or force is applied, the component 308 engages with the shallow-angled surface, providing controlled steering capabilities.

If excessive force is applied, the contact point progresses to the steeper-angled surface 333d, which may convert more of the applied force into lateral movement rather than rotational force. This conversion results in relative movement between the component 308 and the port surface, which the physician can feel as a “slipping” movement. This may create an indication of excessive force before complete disengagement occurs enabling the physician to detect when force thresholds are being approached. If the component 308 begins to “slip” at surface 333d, the physician can still maintain some rotational control through engagement with surface 333e, enabling for adjustment of applied force before complete disengagement.

This configuration provides physical feedback about force application giving physicians an opportunity to respond to initial slippage by reducing applied force, potentially avoiding complete disengagement and maintaining steering control throughout the procedure.

FIG. 3E illustrates the interaction between an extendable component such as the channeling member 338 and channeling distal tip 336 extending through port 332 of the introducer shaft 304. This view demonstrates how the angled surface 333 with lateral angles such as 335a, of port 332 engage with these extendable components. The channeling member 338 extends through port 332 to create paths within the vertebral body. The physician can then rotate and/or move the channeling member 338 by rotating the proximal end of the introducer 304 such that the angled surface 333 of port 332 provide controlled steering at the distal end of the device. The angled surface 333 enable direct steering control at the port location. The port 332 may also include disengagement surface 334 located at the distal end of the port 332, oriented at approximately 90 degrees relative to the central axis 375 of the shaft 304. This disengagement surface 334 is a mechanical clutch because it will not translate a significant amount of rotational force from the introducer to the component. The disengagement surface 334 is a contact surface that the channeling member 338 may engage with when forces exceed certain thresholds. For instance, during normal operation, the channeling member 338 maintains consistent contact with angled surface 333. This contact pattern enables translation of rotational and axial inputs from the handle to the distal end of the channeling member 338. As the channeling member 338 rotates or experiences increasing force exceed predetermined thresholds, the interaction between the channeling member 338 and angled surface 333 changes in a predictable manner. The channeling member 338 begins to shift its contact point distally along one side of the angled surface 333, depending on which direction the introducer 304 is being rotated. As rotational force increases, the channeling member 338 experiences an outward force vector perpendicular to the angled surface 333, the axial component of which causes the contact point to migrate distally. Similarly, when axial force is applied, the angled surface 333 resolves this force into both forward and lateral components, again shifting the contact point distally. If forces continue to increase beyond the threshold the channeling member 338 will continue to shift distally until the channeling member 338 may ultimately disengage from angled surface 333, and the contact point may migrate from the lateral angle 335a of angled surface 333 to disengagement surface 334. This prevents channeling member 338 from failing if the rotational force continued to increase. When the applied forces return below the threshold, the introducer 304 needs to be realigned and the channeling member 338 can return to its original contact position along the proximal regions of the angled surface 333, resuming normal operation.

Once the initial channel is created, port 332 may accommodate various subsequent instruments while maintaining these mechanical principles. Treatment delivery devices, visualization tools, or specialized instruments pass through the port 332, benefiting from the same geometric interaction between component and angled surfaces.

Different configurations of channeling member 338 may be employed based on anatomical requirements and procedural goals. Straight channeling members create linear paths, while curved members generate curved channels within the vertebral body. The angled surfaces of port 332 maintain their force conversion and safety features across these variations through consistent geometric principles that scale with component size and shape.

The geometric configuration of port 332 enables controlled steering without additional mechanical components typically required for torque limitation. Rather than incorporating separate slip clutches or mechanical stops, the angled surface 333 inherently provide both steering control and safety features through their fundamental geometry. This reduces device complexity while maintaining reliable performance characteristics across different procedural applications.

FIG. 3F presents a front view of the port 332 of FIG. 3E with the channeling member 338 positioned at 0 degrees (neutral position) relative to the introducer shaft 304. This perspective reveals lateral angles 335a and 335b of angled surface 333 on opposite sides of the port, as well as the disengagement surface 334 at the base of the port 332. The channeling distal tip 336 extends through the port 332 while the channeling member 338 may remain in contact with the angled surface 333. The elongate shaft portion 354 of the channeling member 338 extends beyond the port 332, creating the working length that reaches target treatment areas within the vertebral body. From this view, the geometric relationship between the port surfaces and the channeling member 338 can be observed in their neutral alignment.

In this position, the channeling member 338 maintains balanced contact with the angled surface 333. The disengagement surface 334 provides a base support for the channeling member 338, creating a three-point contact geometry that stabilizes the component during deployment. When in this neutral position, forces applied to the channeling member 338 are distributed evenly across the contact points on angled surface 333. This balanced force distribution enables directional control during initial deployment while maintaining the mechanical properties that enable for controlled movement and potential disengagement under appropriate conditions.

FIG. 3G illustrates a side view of the introducer shaft 304 with the channeling member 338 positioned to approximately 120 degrees from its neutral position. At this position, the channeling member 338 has shifted its primary contact from the angle 335a to the disengagement surface 334. The elongate shaft portion 354 of the channeling member has moved outwards by a distance 355 from its original position. This outward movement occurs due to the angled geometry of surface 333 converting the rotational force into both rotational and axial components. As the physician applies increasing rotational force or movement, the contact point between the channeling member 338 and the angle surface 333 migrates distally along the surface. When the force exceeds a predetermined threshold, the contact point reaches the junction with disengagement surface 334, causing the observed outward displacement of the elongate shaft portion 354 by distance 355. This displacement distance may vary based on several factors, including the applied force magnitude, the specific angles of lateral angles 335and 335b, the material properties of the introducer 304 and the channeling member 338, and the diameter relationship between the channeling member 338 and the port opening 332. The displacement occurs gradually as force increases, with distance 355 growing proportionally until reaching its maximum when the channeling member 338 fully engages with the disengagement surface 334.

In some embodiments, when force is applied in the opposite direction, the channeling member 338 migrates toward an opposite direction instead. The migration pattern depends on the rotation direction and force application. For instance, when rotated clockwise, the channeling member 338 shifts toward angle 335a of angled surface 333, while counterclockwise rotation cause migration toward angle 335b of angled surface 333. In either case, the geometric principles remain consistent - the angled surface 333 convert excessive force into outward and/or downward movement, while the disengagement surface 334 serves as a mechanical stop that prevents further force transmission. This bidirectional force conversion enables the port 332 to accommodate rotation in either direction while maintaining the same safety and control characteristics.

In some embodiments, the distal tip 336 of the channeling member or probe remains in a relatively stable position when rotational force is applied, rather than moving substantially outward from the introducer shaft. During rotation or force application, the section of the channeling member that contacts the angled surface 333 moves distally to exit ramped zone or angled surface 333. This localized movement at the contact point enables controlled disengagement without causing lateral displacement of the distal tip, maintaining procedural positioning even during force management events. The geometric arrangement of the port 332 and angled surface 333 creates this selective movement pattern, wherein force conversion occurs primarily at the interface point while the distal working end of the channeling member maintains its position within the treatment area.

FIG. 3H provides a front view of the same 120-degree rotation configuration shown in FIG. 3G. From this perspective, the channeling member 338 can be seen contacting the disengagement surface 334 while the channeling distal tip 336 and elongate shaft portion 354 have shifted positions from their neutral alignment. The initial contact zone visible in FIG. 3F has transitioned as the channeling member rotated, with the primary contact point moving from the center of port 332, toward its junction with lateral angle 335a and disengagement surface 334. The disengagement surface 334 now bears a portion of the contact load, altering the force transmission characteristics of the system. The third contact point, previously at opposite side (lateral angle 335b), may be reduced or eliminated in this position, changing the stabilization geometry from the three-point contact seen in the neutral position.

The port geometry accommodates this rotational position while enabling the controlled outward and/or downward movement that prevents component failure. When the physician reduces the applied force, the natural elasticity of the channeling member 338 may cause it to return toward its original position, with the contact points gradually migrating back toward their neutral locations. This reversible displacement enables the physician to maintain procedural continuity even after a disengagement event. The progressive nature of this contact migration provides tactile feedback to the physician, indicating when forces are approaching threshold levels before complete disengagement occurs. This mechanical behavior creates a system where force application correlates with predictable physical responses, enhancing both control and safety during vertebral body procedures.

In some situations, the channeling member 338 will be positioned at approximately 60 degrees relative to its neutral position. At this intermediate angle, the channeling member 338 maintains contact with the lateral angle 335a of angled surface 333 (or 335b depending on the rotation and movement) but does so at a position that is closer to the disengagement surface 334. This 60-degree position represents a transitional state where the contact point has migrated partially along the angled surface 333 but has not yet fully engaged with the disengagement surface 334. At this angle, the elongate shaft portion 354 would show a smaller outward displacement than the distance 355 observed at 120 degrees (e.g. approximately 30-40% of the maximum displacement). Such a 60-degree position demonstrates how the port geometry creates a progressive response to increasing rotational forces.

The port 332 with angled surface 333 can accommodate a range of positions, from the neutral 0-degree position through various intermediate angles to the more extreme rotation of 120 degrees or beyond. Within this range, the mechanical behavior follows consistent geometric principles while producing position-specific effects. In some embodiments, rotation between 0-30 degrees typically maintains primary contact with the angled surface 333 with minimal displacement. In some embodiments, rotation ranging between 30-90 degrees, the contact points progressively migrate toward the disengagement surface 334, with increasing outward and/or downward displacement as rotation increases. In some embodiments, rotation beyond 90 degrees, the channeling member 338 engages more fully with the disengagement surface 334, maximizing the outward and/or downward displacement effect. This graduated response across the rotational range provides predictable mechanical behavior throughout the operational envelope, enabling physicians to develop procedural techniques that maintain control while benefiting from the inherent safety features.

The specific angles at which contact transitions occur depend on the port geometry design parameters. In some embodiments, the angled surface 333 is configured with shallower non-perpendicular angles (5-30 degrees relative to the central axis 375) to extend the rotational range before disengagement occurs. In some embodiments, steeper non-perpendicular angles (30-45 degrees or 30-85 degrees) are employed to trigger earlier disengagement under lower rotational forces. These geometric variations enable the device to be customized for different clinical applications and physician preferences, while maintaining the fundamental mechanical principles that govern force conversion and controlled disengagement.

FIG. 3I illustrates a different embodiment of introducer shaft 304' featuring a different port configuration. This configuration includes port 332' with angled surface 333' that angles inward towards a central axis of the introducer shaft 304'. The angled surface 333' creates a geometric interface where the contact point with channeling member 338 is positioned closer to the central axis of the introducer shaft 304'.

In this embodiment, the angled surface 333' interacts with the channeling member 338 such that rotational forces applied to the introducer shaft 304' or the channeling member 338 are transmitted through the angled surface 333'. The inward orientation of surface 333' concentrates force at localized contact points between the channeling member 338 and angled surface 333', resulting in high force transmission efficiency. While this configuration maintains the geometric principles for potential torque limitation, the increased efficiency of force transmission means that the threshold for disengagement requires substantially higher applied torque compared to other angular configurations. The inward orientation of angled surface 333' creates a mechanical system where the geometrical relationship between the surface and channeling member 338 maximizes force transfer during rotation and movement. The shaft 354 of the channeling member 338 interacts with the angled surface 333' in a manner that enhances force application for steering purposes, while the torque threshold for disengagement exists at forces that exceed typical operational parameters. FIG. 3J illustrates another embodiment of introducer shaft 304" with a port configuration featuring a straight-cut configuration. In this configuration, port 332" includes surface 333" that extends parallel to a central axis of introducer shaft 304" (i.e., at an orientation of 0 degrees), rather than at an angled orientation. The parallel orientation of surface 333" creates a geometric interface with the channeling member 338 that represents an intermediate configuration between outwardly angled surfaces 333 (shown in FIG. 3H) and inwardly angled surfaces 333’ (shown in FIG. 3I). When the channeling member 338 extends through port 332", the shaft 354 interacts with the parallel surface 333" during rotation and movement. This straight-cut configuration transmits rotational forces directly to the channeling member 338. As rotational force is applied and increased, the channeling member 338 may gradually move distally along the surface 333". This gradual movement continues until the channeling member 338 extends beyond the disengagement surface 334", at which point rotational force is no longer effectively transmitted to the channeling member 338.

The open-end at disengagement surface 334" enables the channeling member 338 to continue its extension path beyond the port. In practical application, when sufficient rotational force is applied to the channeling member 338 in this straight configuration, the channeling member may continue to move past the disengagement surface 334". This behavior differs from angled configurations that provide more defined disengagement characteristics, yet still offers a mechanical means by which excessive force application results in reduced force transmission to the channeling member 338.

The ports 332, 332’, 332’’ illustrated throughout the embodiments of FIGS. 3A-3J feature an open disengagement area that enables the channeling member 338 to extend beyond the port structure. This open configuration enables the channeling member 338 to continue its extension path without a defined mechanical boundary at the distal end of the port. The specific interaction between the channeling member and the surfaces of the port determines how rotational and directional forces are transmitted, while the open distal area influences how the channeling member 338 behaves when force thresholds are exceeded. The open port configuration works in conjunction with the various surface orientations to provide different steering characteristics and force management properties during operation.

In some embodiments an introducer cannula may be manufactured from nitinol with a heat-formed J-shaped distal end. This configuration enables creation of aggressive turns within bone tissue through a flexing mechanism that accommodates both insertion and path creation. During operation, the J-shaped distal end can flex to a straightened position during initial access. After the introducer reaches the desired location and the internal stylet is removed, the nitinol material allows the distal end to return to its curved configuration. The introducer can then be advanced forward to create the desired path. The configuration features a small cross-sectional area that minimizes impact on the patient's bone during advancement. A sharp edge incorporated into the distal end facilitates precise path creation within the bone to achieve proper trajectory. The nitinol construction enables the desired angle to be achieved through malleting of the introducer. Following path creation, a straight stylet probe can be advanced through the established channel to reach positions within the vertebral body.

In some embodiment, there may be a configuration that addresses challenges associated with smaller pedicle access instruments through an expandable stent-like design. This configuration protects thin-walled liners that would otherwise be fragile when impacting hard bone during advancement through the pedicle. The configuration may incorporate an expandable and contractible tip that works in conjunction with a thin film liner. The tip can expand to protect the liner during advancement through hard bone, then contract as needed while maintaining the liner in position within the vertebral body for subsequent probe placement. Multiple components comprise the expandable tip section, enabling controlled activation of the protective function. This multi-part construction enables the device to maintain liner protection throughout the procedure while accommodating instrument passage through the protected pathway.

In some embodiments a different approach to curved cannula delivery for smaller gauge applications may be utilized. This configuration may comprise a U-shaped channel to attach to the tip of a curved stylet, creating a maintained pathway for subsequent instrument delivery. The U-shaped channel remains in position after initial path creation, serving as a guide for precise probe placement. This configuration proves particularly valuable for smaller gauge applications by maximizing available space while ensuring accurate instrument tracking regardless of bone conditions. Beyond pathway maintenance, this configuration enables control over treatment delivery patterns. The U-shaped geometry can influence ablation shape toward posterior placement and modify effective electrode size to control ablation dimensions.

These various configurations may be employed independently or in combination based on specific procedural requirements, each providing distinct advantages for channel creation and instrument delivery while maintaining compatibility with the overall system architecture.

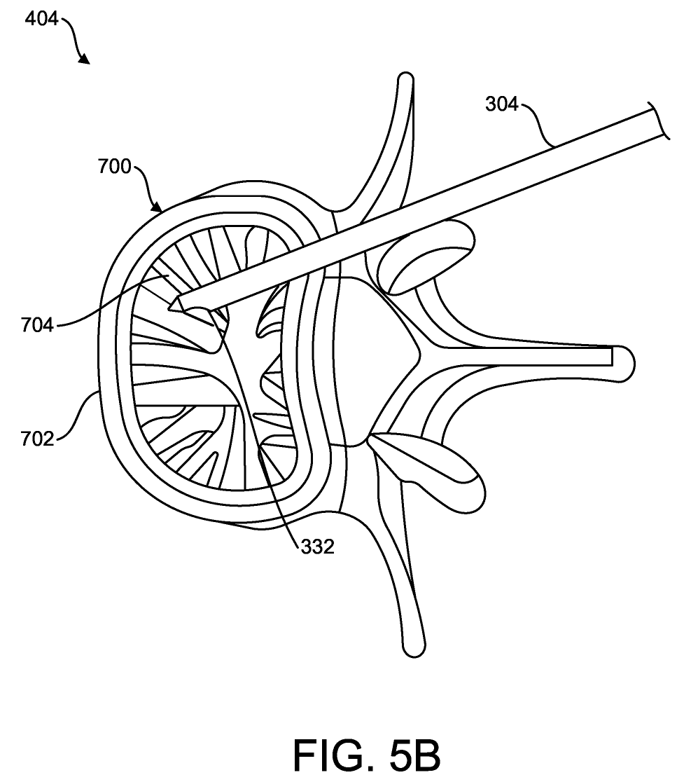

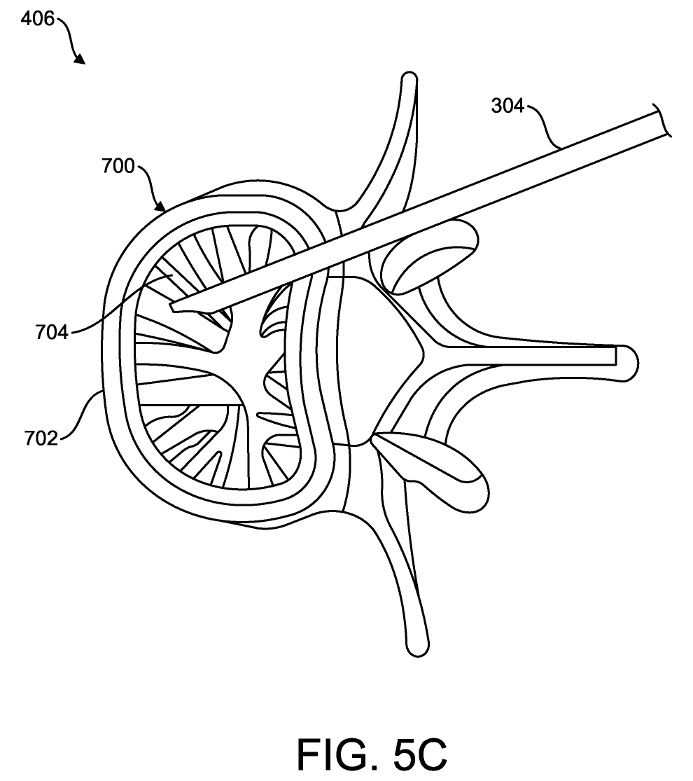

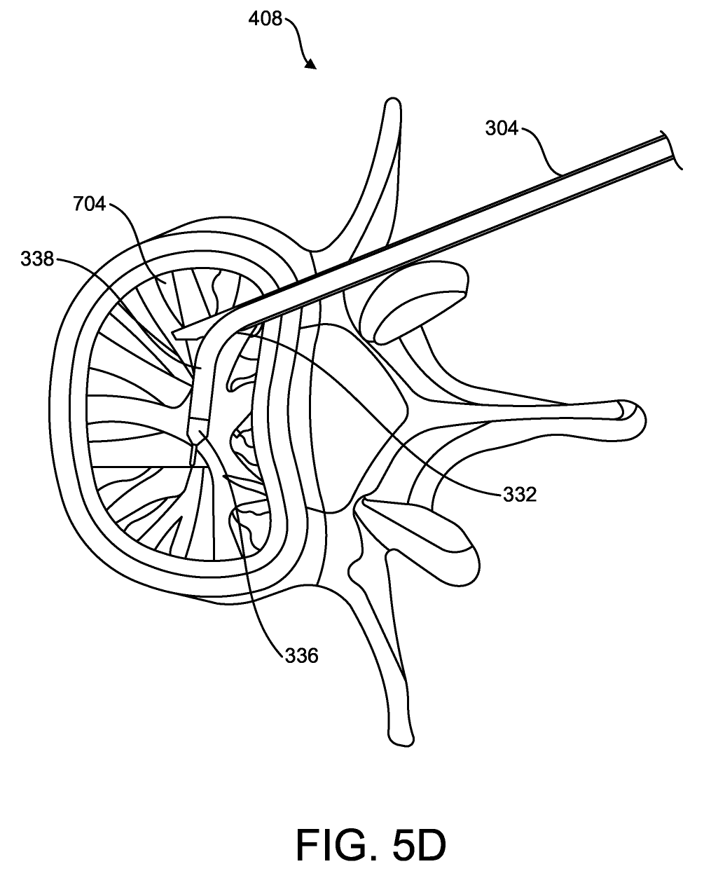

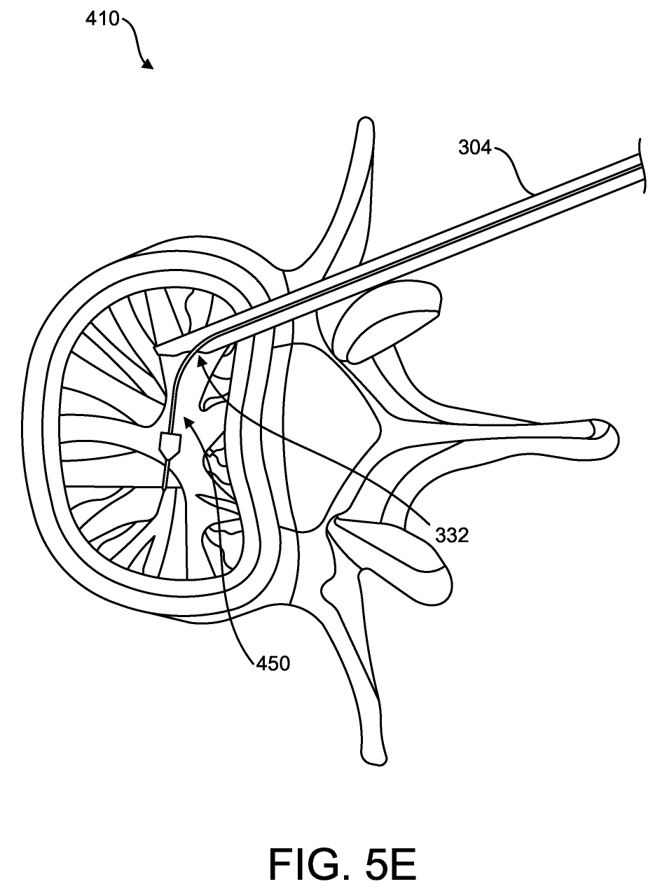

FIG. 4 shows a flowchart of the method 400 of accessing and treating tissue within a vertebral body 700 using a medical device 300. FIGS. 5A-5E show a series of operations of accessing and treating tissue within the vertebral body 700 using the medical device 300. FIGS. 4 and 5A-5E will now be discussed in conjunction with one another, and each operation of the method 400 is illustrated by a corresponding one of FIGS. 5A-5E.

In the illustrated embodiment, the method 400 begins at the operation 402, wherein the medical device 300 is positioned against the vertebral body 700, as shown in FIG. 5A. The medical device 300 can include any suitable combination of the embodiments of components described in the present disclosure. The medical device 300 includes an introducer 304 that is an open-ended hollow shaft. Inside the central channel of the shaft is a chisel 329 with a sharpened tip 330. The device 300 also includes a distal opening 332 with angled surfaces positioned near the tip 330 that connects with the interior cavity of the introducer shaft 304.

At the operation 404, a portion of the medical device 300 is driven into the vertebral body 700 (e.g., by malleting the medical device 300), through the cortical bone region 702, and into the cancellous bone region 704 far enough that the distal end of the introducer 304 is located in the cancellous bone region 704, as shown in FIG. 5B.

At the operation 406, the chisel 329 is removed from the introducer 304, as shown in FIG. 5C.

At operation 408, an extendable component such as a channeling member 338 is extended into the cancellous bone region 704 from the interior of the introducer shaft 304 through the opening 332, as shown in FIG. 5D. As the channeling member 338 advances, the angled surfaces of port 332 engage with the channeling member to provide controlled steering at the distal end of the device. The physician can rotate the introducer shaft 304 and move the channeling member 338 to create desired paths within the vertebral body, with the port geometry converting rotational input into controlled movement while preventing excessive force application through its angular configuration.

During path creation, if forces rotational forces exceed predetermined thresholds, the angled surfaces of port 332 enable the channeling member 338 to disengage rather than experience mechanical failure within the vertebral body. This geometric safety feature maintains device integrity throughout the channeling process while enabling precise path creation. After establishing the desired channel, the channeling member 338 can be removed from the introducer 304.

At operation 410, the elongate shaft portion 354 (shown in FIG. 3J) is withdrawn, leaving the core 450 of the channeling member 338 (shown in FIG. 5D) at the treatment location, as shown in FIG. 5E. In some embodiments, the core 450 serves as a guidewire over which other medical instruments can be advanced to the treatment location. Such medical instruments can include, for example, energy delivery probes (e.g., a bipolar radiofrequency ablation probe), fluid delivery instruments, or other therapeutic tools configured to treat the target site through the established channel. In some embodiments, the core 450 serves as an electrode for an energy delivery probe that includes another electrode. In some embodiments, the channeling member 338 can function as a stylet. In some embodiments, the angled surfaces maintain their mechanical properties during instrument exchange, providing the same controlled positioning and safety features for the treatment devices as for the channeling member 338.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. Amedical device comprising:
an elongate shaft defining a central channel extending from a proximal end to a distal end of the shaft, the shaft defining a distal port in communication with the central channel;
a handle disposed at the shaft proximal end;
a channeling member disposed within the central channel of the shaft and configured to extend through the port; and
wherein the port comprises an angled surface oriented at a non-perpendicular angle relative to a central axis of the elongate shaft and an end surface oriented at a perpendicular angle relative to the central axis, wherein the non-perpendicular angle of the surface is configured to facilitate disengagement of the channeling member from the angled surface and facilitate engagement of the channeling member with the end surface when a force exceeds a predetermined threshold.

2. The medical device of claim 1, wherein:
the channeling member defines a proximal end and a curvable distal end;
the distal end of the channeling member is movable between:
a first position, at which the distal end of the channeling member is disposed within the central channel proximal of the port, and a second position, at which the distal end of the channeling member is disposed exterior of the central channel and the port; and
wherein the channeling member is configured to channel through cancellous bone in a vertebral body.

3. The medical device of claims claim 1, wherein the channeling member comprises a curvable distal portion configured to deflect within a vertebral body when extended through the port.

4. The medical device of claim 1, wherein the angled surface is configured to distribute rotational and axial forces to change the point of contact between the channeling member and the elongate shaft.

5. The medical device of claim 1, wherein the angled surface is further configured to provide controlled directional movement of the channeling member.

6. The medical device of claim 1, wherein the non-perpendicular angle of the surface is configured to limit or increase torque transmission to the channeling member based on the geometric relationship between the angled surface and the channeling member.

7. The medical device of claim 1, wherein the non-perpendicular angle is configured to convert a portion of force into an axial force and/or rotational force component that facilitates controlled disengagement of the channeling member.

8. The medical device of claim 1, wherein the port is configured to enable complete withdrawal of the medical device from a vertebral body upon disengagement of the channeling member.

9. The medical device of any of claim 1, wherein the non-perpendicular angle is between 10 degrees and -10 degrees relative to the central axis of the elongate shaft.

10. The medical device of claim 1, wherein the angled surface is configured to:
maintain engagement with the channeling member when applied force is below the predetermined threshold;
gradually cause a contact point between the channeling member and the angled surface to shift in a distal direction as applied force approaches the predetermined threshold; and
enable re-engagement of the channeling member when applied force is reduced below the predetermined threshold.

11. The medical device of claim 1, wherein the angled surface extends circumferentially around a portion of the elongate shaft to define a controlled engagement region.

12. The medical device of claim 1, wherein the angled surface transitions steering control from the proximal end to the distal end of the elongate shaft.

13. The medical device of claim 12, wherein the distal steering control reduces torsional stress accumulation of the channeling member within the central channel.

14. The medical device of claim 1, wherein the port comprises a geometry that provides increasing mechanical resistance as the force approaches the predetermined threshold.

15. The medical device of claim 1, wherein the angled surface comprises a geometric configuration that maintains directional control while preventing structural failure of the channeling member.

16. A system for treating tissue within a vertebral body, the system comprising:
an elongate shaft defining a central channel extending from a proximal end to a distal end of the shaft, the shaft defining a port in communication with the central channel, the shaft further comprising a distal tip;
a handle disposed at the shaft proximal end;
a channeling member disposed within the central channel of the shaft and configured to extend through the port, wherein:
the channeling member defines a proximal end and a curvable distal end,
the distal end of the channeling member is movable between:
a first position, at which the distal end of the channeling member is disposed within the central channel proximal of the port, and
a second position, at which the distal end of the channeling member is disposed exterior of the central channel and the port, and
the channeling member is configured to channel through cancellous bone in the vertebral body;
wherein the port comprises an angled surface oriented at a non-perpendicular angle relative to a central axis of the elongate shaft, wherein:
the angled surface is configured to engage the channeling member during rotational movement of the shaft to translate rotational force to the channeling member, and
the non-perpendicular angle of the surface is configured to facilitate disengagement of the channeling member from the port when the rotational force exceeds a predetermined threshold.

17. The system of claim 16, wherein the angled surface proximate to the distal end of the shaft is configured to reduce mechanical stress concentration on the channeling member during rotational movement.

18. The system of claim 16, wherein the non-perpendicular angle is configured to convert a portion of rotational force into an axial force component that facilitates controlled disengagement of the channeling member.

19. The system of claim 16, wherein the angled surface transitions steering control from the proximal end to the distal end of the elongate shaft to reduce torsional stress accumulation of the channeling member within the central channel.

20. A method of treating tissue within a vertebral body of a patient, the method comprising:
providing a medical device comprising:
a cannula defining a central channel extending from a proximal end to a distal end of the cannula, the cannula defining a port proximate the distal end and in communication with the central channel, the port comprising an angled surface oriented at a non-perpendicular angle relative to a central axis of the cannula and an end surface oriented at a perpendicular angle relative to the central axis;
a handle disposed at the cannula proximal end, the handle comprising a first actuator;
a stylet disposed within the central channel of the cannula, the stylet defining a proximal end and a distal end, wherein the stylet is coupled with the first actuator and axially movable, in response to actuation of the first actuator, such that the distal end of the stylet is advanceable out of the port and retractable into the central channel of the cannula; and
inserting the cannula of the medical device through cortical bone of the vertebral body into a cancellous bone region of the vertebral body;
actuating the first actuator to advance the distal end of the stylet out of the port;
rotating the cannula to steer the stylet within the vertebral body, wherein the angled surface of the port engages the stylet to translate rotational force to the stylet, and the non-perpendicular angle facilitates disengagement of the stylet from the angled surface when rotational force exceeds a predetermined threshold;
creating a channel within the vertebral body using the stylet;
advancing a treatment device through the channel to a treatment location within the vertebral body; and
delivering treatment to the tissue within the vertebral body via the treatment device.
